(19) United States
(12) Patent Application Publication
(10) Pub. No.: US 2013/0104910 A1
(43) Pub. Date: May 2, 2013

(54) APPARATUS AND METHOD FOR MAINTAINING STERILE FIELD

(71) Applicants: Todd Kerner, Columbus, OH (US); John Bartsch, Culumbus, OH (US)

(72) Inventors: Todd Kerner, Columbus, OH (US); John Bartsch, Culumbus, OH (US)

(21) Appl. No.: 13/285,765

(22) Filed: Oct. 31, 2011

(51) Int. Cl. A61B 19/08

(52) U.S. Cl. 128/853

(57) ABSTRACT

A drape for maintaining a sterile field for a real-time guided medical procedure (such as with ultrasound guidance) may include a contact film base having an aperture therethrough. A shield may project outwardly from the base and may surround the aperture.

### BACKGROUND OF THE INVENTION

The present invention generally relates to apparatus and methods for maintaining a sterile field when employing a percutaneous medical device.

Certain medical procedures such as intra-articular joint injections, peripheral nerve blocks, vascular access, and soft tissue biopsies, often rely upon ultrasound imaging to determine the location of the percutaneous device within a patient's body.

A sterile field must be maintained around an entry point of a percutaneous device. When utilizing ultrasound for guidance for percutaneous procedures, the ultrasound probe is usually placed as close as possible to the sterile entry point so as to assure clear, real-time guidance through all tissues transected by the device. This sterile field must remain intact whenever ultrasound is used in the vicinity of the entry point. Consequently, guidance devices such as ultrasound probes used in such applications must be provided with sterile covering. Additionally, materials used to enhance the operation of the guidance devices also must be sterile. For example, gel material used in conjunction with a sterile ultrasound probe must also be sterile because the gel may migrate to the entry point of the percutaneous device during a procedure.

As can be seen, there is a need for maintaining a sterile field for guided employment of a percutaneous medical device without incurring a need to employ sterile guidance devices.

### SUMMARY OF THE INVENTION

In one aspect of the present invention, a drape for maintaining a sterile field may comprise: a contact film base having an aperture therethrough; a coupler, and a shield projecting outwardly from the base and surrounding the aperture.

In another aspect of the present invention, a method for performing a medical procedure may comprise the steps of: applying a sterile drape on a patient's skin with an aperture of the drape overlying a desired entry point; placing a medical device into an outwardly projecting shield of the drape; and performing the procedure while maintaining the medical device in the shield.

These and other features, aspects and advantages of the present invention will become better understood with reference to the following drawings, description and claims.

### BRIEF DESCRIPTION OF THE DRAWINGS

FIG. 1 is a perspective view of an operational aspect of a sterile-field drape in accordance with an embodiment of the invention;

FIG. 2 is a perspective view of the sterile-field drape of FIG. 1;

FIG. 3 is an elevation view of the sterile-field drape of FIG. 2;

FIG. 4 is a cross-sectional view of the sterile-field drape of FIG. 1;

FIG. 5 is a cross-sectional view of the operational aspect of FIG. 1 taken along the lines 5-5; and

FIG. 6 is a flow chart of a method for performing a medical procedure in accordance with an embodiment of the invention.

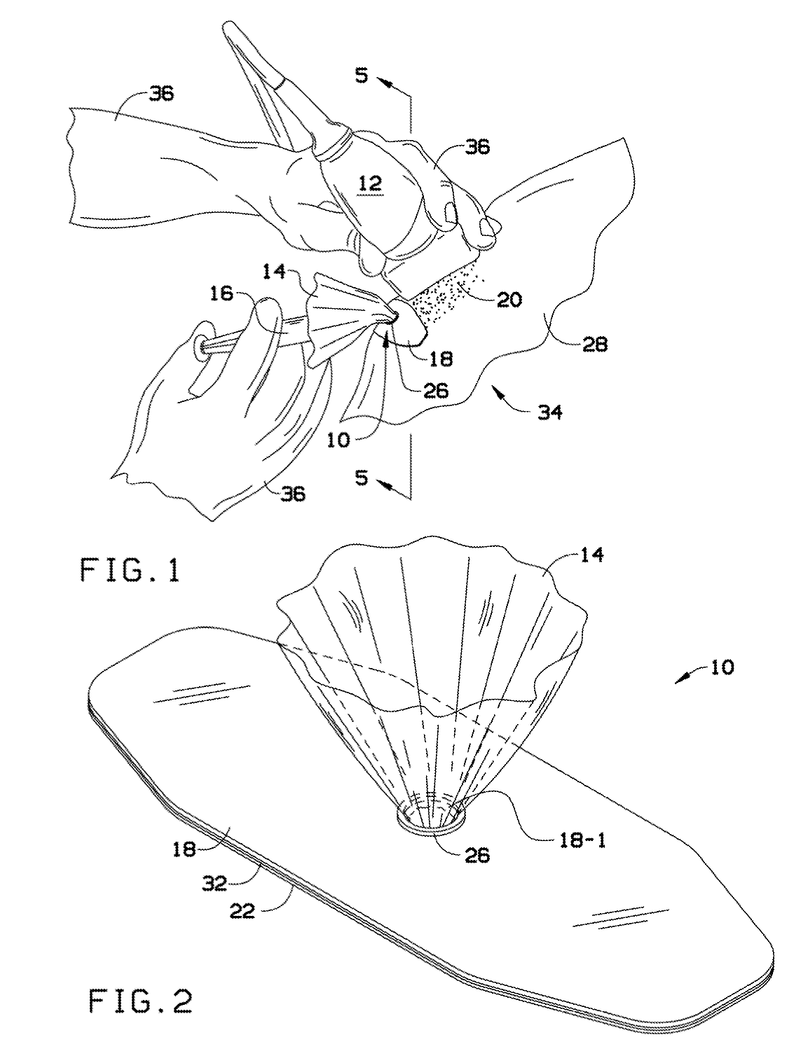

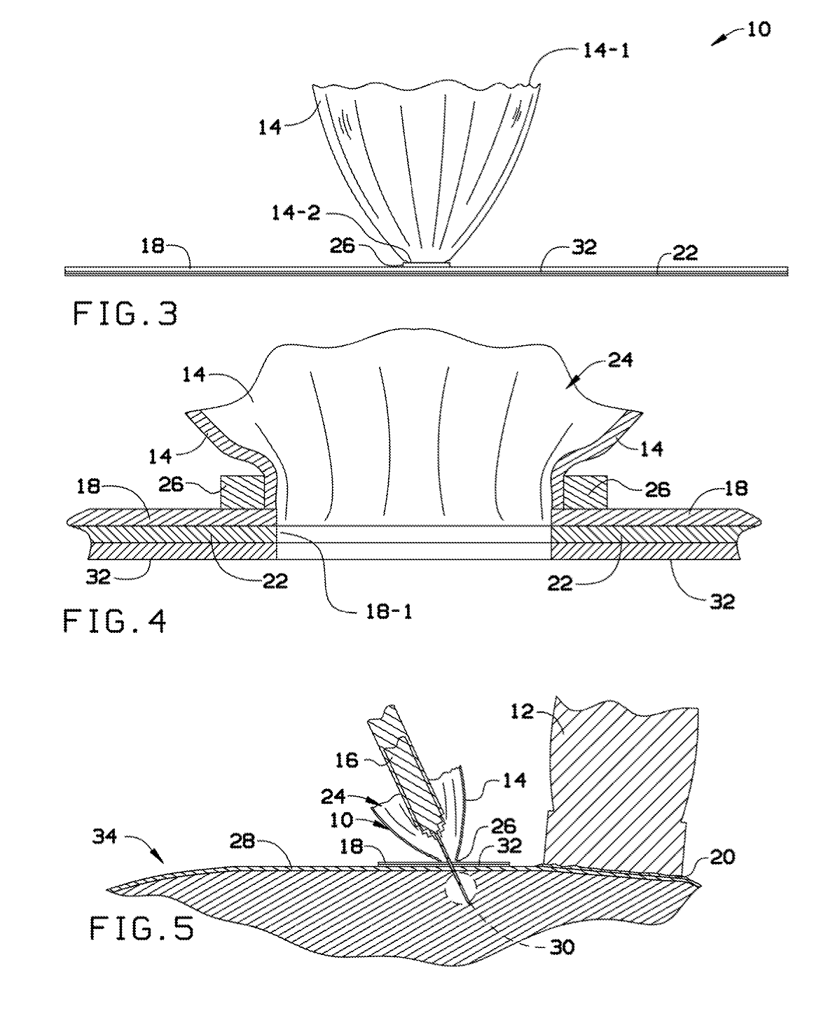

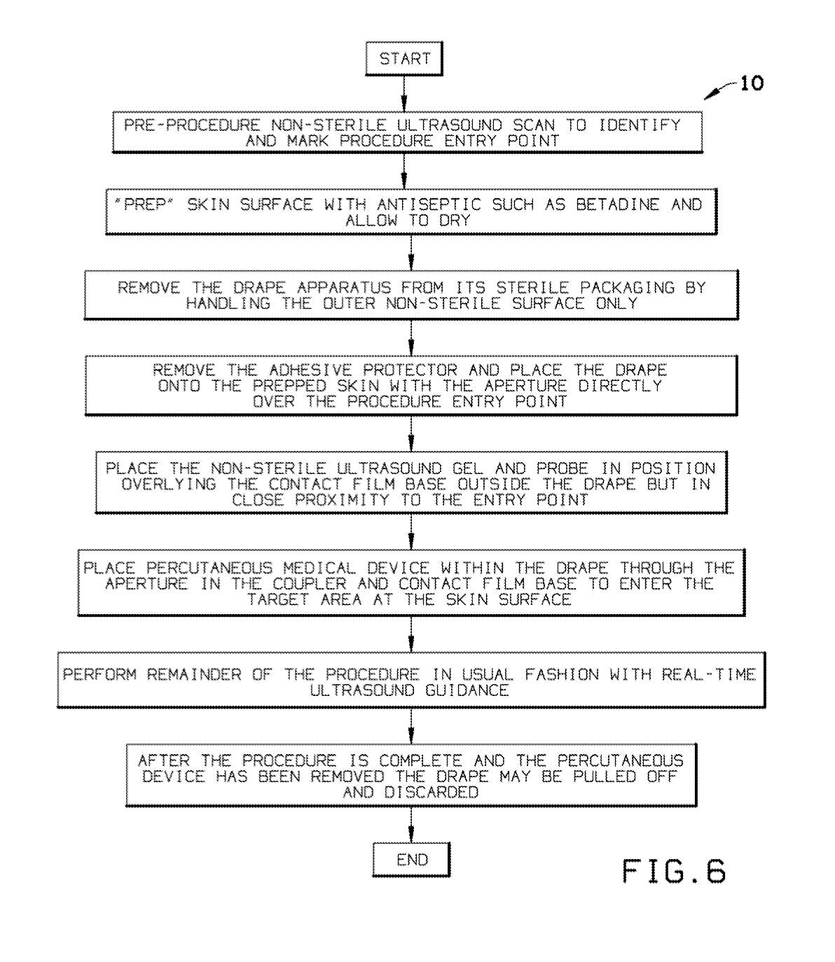

### DETAILED DESCRIPTION OF THE INVENTION

The following detailed description is of the best currently contemplated modes of carrying out exemplary embodiments of the invention.

The description is not to be taken in a limiting sense, but is made merely for the purpose of illustrating the general principles of the invention, since the scope of the invention is best defined by the appended claims.

Various inventive features are described below that can each be used independently of one another or in combination with other features.

Broadly, embodiments of the present invention generally provide a sterile-field drape that may be positioned on a patient so that a percutaneous medical device may be operated through an outwardly projecting shield.

Referring to the Figures, it may be seen that an exemplary sterile-field drape 10 may comprise a shield 14, a coupler 26, and an attachment contact film base 18.

The contact film base 18 may be provided with a layer 22 of adhesive and an adhesive protector 32. The contact film base 18 may be polyurethane film or aromatic polyether polyurethane film. The material of the contact film base 18 may provide durable, intimate contact with the skin and allow unimpeded transmission of the ultrasound beam. The contact film base 18 may be pliable so that it can adhere to irregular skin surfaces. Finally, the contact film base 18 may be impermeable to liquids because it needs to assure a sterile barrier from the surrounding ultrasound gel.

The shield 14 may be constructed from a thin, pliable film material such as polyester film or injection molded silicone. The shield 14 may be frustro-conical in shape to accommodate a wide variety of percutaneous devices. In other words, an upper extremity 14-1 of the shield may have an opening larger than an opening in a lower extremity 14-2 of the shield 14. The shield's size may vary based upon the location and application. For example, for a procedure on a finger this could be about 4 to 6 cm. For a procedure on an abdomen, the outer width could be 8 to 12 cm. The shape may enable the guidance device 12 (such as the ultrasound probe) to be placed as close as possible to the sterile target area but may allow for ease of manipulation of the percutaneous medical device with minimal hindrance while maintaining good visualization at all times. The shield 14 may project out from an aperture 18-1 at adjustable angles high enough to maintain a sterile field around the target area (i.e. always prevents non-sterile ultrasound-probe gel from spilling into the sterile target area). The shield 14 may also be constructed with an embedded light spring (plastic or metal) in the same frustro-conical shape. This would allow the entire sterile-field drape 10 to be packaged in a collapsed form.

A coupler 26 may hold the shield 14 in position on the contact film base 18. The coupler 26 may be constructed of liquid silicone injection molding or polyurethane that is either press fit or glued. Alternatively, all components may be one solid molded material to conjoin the shield 14 and the contact film base 18. The coupler 26 may be of appropriate durometer to enable the clinician to manipulate the percutaneous device within the shield 14 through a wide range of angles without compromising the integrity of the sterile field drape 10 at the point where the shield 14 narrows to the aperture 18-1 on the target site. The shield 14 and the coupler 26 may surround the aperture 18-1 in the contact film base 18. The aperture 18-1 may be any size large enough to accommodate a percutaneous device 16 but small enough for the shield 14 to maintain the sterile barrier. Smaller apertures would be preferred as the optimal procedure would be one where the ultrasound probe is placed close to the entry point of the percutaneous medical device 16. This would assure continuous visualization of the percutaneous medical device through all tissues transected. For typical applications, this aperture 18-1 may be about 5 to 10 mm in diameter.

In operation, the sterile field drape 10 may be placed on a patient 34 so that the contact film base 18 is adhesively attached to a sterilized portion of a patient's skin 28. The contact film base 18 may be positioned so that the aperture 18-1 overlies a desired entry point for the percutaneous medical device 16. A practitioner 36 may place the percutaneous medical device 16 within the shield 14 and insert the device 16 into the entry point. A guidance device 12 such as an ultrasound probe may be placed on the contact film base 18 which overlies the patient's skin 28 in close proximity to the entry point so that the practitioner 36 may more readily guide the device 16 to a desired target area 30.

The shield 14 of the sterile field drape 10 may preclude contact between the guidance device 12, such as the ultrasound probe, and the percutaneous medical device 16. Thus, there may be no need to maintain the guidance device 12 in a sterile state. Moreover, if the guidance device 12 needs a gel application 20 for proper operation, the practitioner 36 may employ non-sterile gel because the shield 14 may also preclude contact between the gel 20 and the percutaneous medical device 16.

It may also be noted that, because the shield 14 may be pliable, the percutaneous medical device 16 may be readily manipulated by the practitioner 36 while the shield 14 may be displaced in conformity with the position of the percutaneous medical device 16.

While the sterile-field drape 10 has been described herein as being particularly useful in procedures using of a first medical instrument (e.g., the percutaneous device 16) inside the shield 14 and a second instrument (e.g., the probe 20) outside the shield 14, it should be noted that the sterile-field drape 10 may be useful for any application in which it is desired to expediently produce and maintain a sterile field of limited size.

It should be understood, of course, that the foregoing relates to exemplary embodiments of the invention and that modifications may be made without departing from the spirit and scope of the invention as set forth in the following claims.

What is claimed is:

1. A drape for maintaining a sterile field comprising:
a contact film base having an aperture therethrough;
and
a shield projecting outwardly from the base and surrounding the aperture.

2. The drape of claim 1 wherein the contact film base is impermeable to liquids.

3. The drape of claim 1 wherein the shield projects out from the aperture at adjustable angles.

4. The drape of claim 1 wherein the shield is frustro-conical in shape.

5. The drape of claim 1 wherein the shield includes an embedded light spring.

6. The drape of claim 1 wherein the contact film base has an adhesive attachment layer.

7. The drape of claim 1 wherein the contact film base is pliable.

8. The drape of claim 1 wherein the contact film base is a material that will not impede ultrasound beam transmission.

9. A method for performing a medical procedure comprising the steps of:
applying a sterile-field drape on a patient's skin with an aperture of a contact film overlying a desired entry point;
placing a medical device into an outwardly projecting shield of the drape; and
performing the procedure while maintaining the medical device in the shield.

10. The method of claim 9 further comprising the step of employing a guidance device positioned outside of the shield, to guide the percutaneous medical device.
